(19) United States
(12) Patent Application Publication
(10) Pub. No.: US 2004/0265359 A1
(43) Pub. Date: Dec. 30, 2004

(54) Oral delivery formulations of L-glutamine

(76) Inventors: Gordon S. Sacks, Columbus, WI (US); Aimee B. Sacks, Columbus, WI (US)

Correspondence Address: FULBRIGHT & JAWORSKI L.L.P., A REGISTERED LIMITED LIABILITY PARTNERSHIP, SUITE 2400, 600 CONGRESS AVENUE, AUSTIN, TX, 78701-3271, US

(21) Appl. No.: 10/824,767

(22) Filed: Apr. 15, 2004

Related U.S. Application Data
(60) Provisional application No. 60/463,561, filed on Apr. 17, 2003.

(51) Int. Cl. A61K 47/00; A61K 31/198

(52) U.S. Cl. 424/439; 424/440; 514/563

(57) ABSTRACT

The present invention is directed to solid and semi-solid formulations of the amino acid L-glutamine. Particular formulations include popsicles, lollipops, oral strips and lozenges, optionally including flavor agents and/or anesthetics and media. Also provided are methods for the treatment of mucositis, particularly that associated with chemo- and radiotherapy for cancer patients.

### BACKGROUND OF THE INVENTION

1. Field of the Invention

The present invention relates generally to the fields of pharmacology, medicine and oncology. More particularly, it concerns formulations and methods for treating oral inflammation using amino acids.

2. Description of Related Art

Cytotoxic radio- and chemotherapy often produce a variety of adverse side effects including nausea, weakness, fatigue, hair loss and inflammation. Inflammation of mucosal tissues (mucositis) of the mouth and gastrointestinal tract is a particular problem, both because of patient discomfort, and due to the secondary effects on patient nutrition. Weight loss and dehydration often follow, which is further complicated when prior surgery has caused problems in mastication and swallowing.

One agent that appears effective in dealing with mucositis is L-glutamine, an amino acid known to be important in cell maintenance, division and repair. Studies show that glutamine, as an adjuvant therapy, also is beneficial in decreasing tumor growth, as well as exerting positive effects on enterocolitis and also mucositis. Many studies also show that prolonged contact of glutamine to mucosal membranes is necessary for the manifestation of its beneficial effects, limiting the value of oral “swish” protocols.

Unfortunately, glutamine is poorly soluble in water. Thus, administration of glutamine to cancer patients through diet is difficult both because of associated pain during swallowing, and because of the relatively large volumes of liquid required for the proper dose. Thus, improved methods for glutamine deliver to patients suffering from oral inflammation is required.

### SUMMARY OF THE INVENTION

Thus, in accordance with the present invention, there is provided a drug formulation designed for oral release comprising (a) L-glutamine; and (b) a solid or semi-solid edible medium that dissolves upon oral contact. The formulation may be provided in a unit dose of about 20 to about 40 grams L-glutamine. The formulation may further comprise a flavor agent, an antiseptic or an anesthetic. The medium may be water, a gel or pudding, or a mixture thereof. The formulation may be provided as a lozenge, a dissolvable strip, a lollipop or a popsicle. The formulation may dissolve in response to an aqueous environment, may dissolve at or near normal body temperature, or may dissolve in response to a combination of an aqueous environment and temperature at or near normal body temperature. In particular, the formulation may be provided as a popsicle comprising pudding, water, and a flavor agent. The formulation may be frozen, e.g., solid at a temperature below about 0° C.

In another embodiment, there is provided a method of treating oral inflammation comprising administering to a subject in need thereof a drug formulation designed for oral release comprising (a) L-glutamine; and (b) a solid or semi-solid edible medium that dissolves upon oral contact. The formulation may further comprise a flavor agent, an antiseptic or an anesthetic. The medium may be water, a gel or pudding, or a mixture thereof. The formulation may be provided as a lozenge, a dissolvable strip, a lollipop or a popsicle. The formulation may dissolve in response to an aqueous environment, may dissolve at or near normal body temperature, or may dissolve in response to a combination of an aqueous environment and temperature at or near normal body temperature. In particular, the formulation may be provided as a popsicle comprising pudding, water, and a flavor agent. The oral inflammation may be chemotherapy-induced or radiation-induced.

In yet another embodiment, there is provided a storage stable drug formulation comprising L-glutamine frozen in a flavored medium. The L-glutamine may be stable for about 3 to about 12 months. The formulation may be provided in a unit dose of about 20 to about 40 grams L-glutamine.

In still yet another embodiment, there is provided a kit comprising (a) L-glutamine; and (b) a flavor agent. The kit may further comprise one or more of (c) an edible medium; (d) an anesthetic; (e) a handle; and/or (f) a mold.

### DESCRIPTION OF ILLUSTRATIVE EMBODIMENTS

I. The Present Invention

As discussed, above, chemo- and radiotherapy is a major cause of patient discomfort, a significant aspect of which is oral inflammation. L-glutamine, a natural amino acid, has been shown effective at combating such inflammation, but its delivery is somewhat complicated by insolubility in water, and the discomfort associated with oral intake of large liquid volumes.

Thus, the present invention seeks to take advantage of various oral delivery forms in the administration of glutamine to patients suffering from mucositis. Of particular interest are the use of popsicles, lollipops and lozenges. These drug formulations offer some important advantages. First, they are familiar to most patients and well received, being substances normally associated with pleasure. This achieves higher patient compliance. Second, lollipops and popsicles in particular can permit the physician and/or patient to control the dosage of the drug administered to the patient, thereby resulting in dose-to-effect drug administration. Third, lollipops and popsicles allow the patient to self-medicate and exert control over the dosage received in order to diminish feelings of discomfort or pain.

These important advantages are available because relatively small amounts of glutamine may be delivered to a patient substantially continuously, and administration of the glutamine may be halted at any time. This not only allows a physician to monitor a patient's condition so that a particular effect is obtained and maintained, but also provides an important safety benefit. It is much less likely that a patient receiving medication in accordance with the drug formulations of the present invention will become overdosed since the dose builds relatively slowly until a desired effect is achieved. Further, if a patient becomes slightly overdosed, it is likely that the patient will cease administration before becoming seriously overdosed and/or the physician or other medical personnel will observe the situation and intervene.

II. Mucositis

Mucositis is defined as inflammation of the mucous membrane. When it occurs in the oral cavity, it is further defined as stomatitis. There are a variety of causes for stomatitis, one of which is surgery to the head and neck area. However, chemotherapy and radiotherapy present the major challenge, especially since most cancer patients are faced with other health issues, including loss of appetite, weight loss and malnutrition.

Acute oral mucosal reaction (mucositis) is caused by radiation- or drug-induced cell division (mitotic) death of the basal cells in the oral mucosa. When the radiation or chemotherapy is delivered at a rate equivalent to or greater than the ability of the oral mucosa to regenerate, then severe mucositis will be seen. Oral microorganisms also probably play a role in aggravating this condition. Late or post-treatment-induced atrophy and telangiectasis of the mucosa often increase the risk for pain and/or necrosis. Management may require interruption of therapy, topical anesthetics (discussed above) may be of some value, but the pain often requires systemic analgesic drugs. Since infections may be associated, antimicrobial/antifungal agents should also be considered.

Taste buds, which occur primarily in the tongue papillae, are very sensitive to radiation. Because of their location, it is difficult to exclude them from radiation for most oral cancers, and are impossible to exclude from chemotherapy. Therefore, may also patients develop a partial or, most usually, complete loss of taste during treatment. While cells comprising taste buds usually regenerate within four months after treatment, the degree of long-term impairment of taste is quite variable.

III. L-Glutamine and Inflammation

Glutamine is a neutral, non-essential amino acid. It is also the most abundant amino acid, comprising about 60% of the total free amino acid pool. Glutamine contains two nitrogen moieties, and as such, it may also be one of the most versatile amino acids. It is critical in nitrogen transport and acts as a primary fuel for rapidly dividing cells (as efficient as glucose). Oxidized glutamine provides substrate for the synthesis of purines and pyrimidines needed for DNA, RNA, and mRNA. There is evidence that glutamine serves a cyto-protective role as well.

During increased metabolic stress, glutamine is released from skeletal muscle, and intracellular glutamine concentrations fall by more than 50%. Although the body can synthesize glutamine, it is considered a conditionally essential amino acid during periods of catabolism, i.e., when the glutamine synthesis rate cannot keep up with the higher requirements during stress. Some studies suggest that during periods of stress, 15-35 grams of supplemental glutamine may be sufficient to preserve muscle glutamine, maintain gut integrity, provide fuel for cells with rapid turnover and improve overall nitrogen balance.

Research into the use of glutamine as an adjuvant therapy in the areas of sepsis, burns, trauma, inflammatory bowel disease, reperfusion injury and AIDS is ongoing. Similarly, there has been growing interest over the past 20 years in defining what role glutamine may play in cancer treatment, therapy tolerance and symptom management. In addition, the role of glutamine as an immunomodulator has been emerging. For example, there is evidence that tumor growth is inversely related to host glutamine stores, and that cancer cachexia is marked by massive host skeletal glutamine depletion. In vitro evidence of the dependence of tumor growth on glutamine has somewhat deterred its use in the clinical setting, but in vivo evidence suggests that supplemental glutamine actually decreases tumor growth by upregulating the immune system.

One clear area of utility for glutamine is as an anti-inflammatory. The use of glutamine in this context has been explored in wound healing, Inflammatory Bowel Disease (IBD), various forms of peritonitis, and athletes recovery from excessive muscle stress. In addition, glutamine has been applied to treat chemo- and radiotherapy induce oropharyngeal inflammation in cancer patients, in particular those undergoing bone marrow transplants. It is in this context that the present invention will find particular utility, being designed for oral delivery.

There is some evidence that glutamine can have an adverse effect on patients with kidney or liver disease. Thus, the administration may be contra-indicated for these individuals, or at least more closely monitored.

IV. Formulations

A. Forms

The present invention may employ various solid or semi-solid oral delivery vehicles. In particular, the inventors contemplate the use of popsicles, lollipops and lozenges. The features of each delivery form are discussed in detail below.

i. Popsicle

The popsicle was invented by an eleven-year-old Frank Epperson in 1905. Epperson was only 11 years old when he invented the “Epsicle.” He had left fruit flavored punch outside with a stir stick in it. The drink froze overnight to the stick, and rather than dispose of it, he attempted to lick the frozen drink, which tasted good. It took 18 more years for Epperson to apply for a patent for “frozen ice on a stick” called the “Epsicle Ice Pop,” which his children re-named “popsicle.” Epperson sold his rights in the popsicle to the Joe Lowe Company of New York in 1925. Good Humor now owns the rights to the Popsicle name.

In one form, the present invention will utilize a popsicle for the oral delivery of glutamine. Everyone is familiar with a popsicle, which comprises a frozen, aqueous solution in which a handle or stick is embedded. The patient will such or lick the popsicle, melting the frozen solution, thereby releasing the glutamine.

Use of a popsicle to delivery glutamine has a number of advantages. First, it achieves one primary goal in oral glutamine therapy, namely, to provide prolonged contact of the oral mucosa with the drug. Second, it allows for a more concentrated delivery of glutamine, as the drug need not be completely dissolved in the base solution (e.g., water and/or pudding). Third, the low temperature has a soothing effect on the patient, providing an additional, temporary anesthetic effect. Fourth, as with other formulations of the present invention, it allows for straightforward and safe self-medication. And fifth, it permits withdrawal of treatment simply by denying the popsicle to the patient.

ii. Lollipop

In another embodiment, L-glutamine may be delivered orally in the form of a “lollipop” or “sucker.” Generally, lollipops and suckers are defined by a solid matrices into which a drug has been dispersed. They are solid or semi-solid at room temperature, and are dissolved by contact with an aqueous environment, i.e., the mouth. Dissolution of the matrices (and hence, release of the drug) may be enhanced by the increased temperature (as compared to ambient or room temperature) of the mouth. Lollipops can be a convenient vehicle for administering a drug to a patient, and differ from a lozenge in that the lollipop can be temporarily removed from the patient's mouth. This enables the patient to communicate orally when necessary, and to control the duration and extent of delivery. Lollipops have an advantage over popsicles in that they are easily transported without refrigeration.

U.S. Pat. No. 4,671,953 discloses a drug dispersed within a carbohydrate mass or other suitable matrix. The drug-containing carbohydrate mass is given to a patient to suck on so that the drug will be released into the patient's mouth as the carbohydrate mass dissolves. The drug is then absorbed through the mucosal tissues of the mouth, as well as the pharyngeal and esophageal areas. See also U.S. Pat. No. 5,484,602.

U.S. Pat. No. 6,159,492 discloses another medicated lollipop. The lollipop includes a stick having opposed ends. A medicated candy is securely attached to one end of the stick. The apparatus further includes a safety container having a closed bottom and a continuous sidewall extending upwardly from the closed bottom. Portions of the sidewall remote from the bottom define an open mouth. The mouth includes a locking structure extending thereabout. For example, the locking structure may include an annular rim or an array of threads. The mouth of the container is sufficiently wide to enable the medicated candy to be passed through. Additionally, the mouth of the container is spaced sufficiently from the bottom wall of the container to permit the entire lollipop, including the candy and the stick to be received in the container.

iii. Lozenge

Solid lozenges are well known in the drug delivery field. In addition to the matrix and drug, a lozenge may contain other ingredients known in such dosage forms such as acidity regulators, opacifiers, stabilizing agents, buffering agents, flavorings, sweeteners, coloring agents and preservatives. For example, solid formulations may be prepared as lozenges by heating the lozenge base (e.g., a mixture of sugar and liquid glucose) under vacuum to remove excess water and the remaining components are then blended into the mixture. The resulting mixture is then drawn into a continuous cylindrical mass from which the individual lozenges are formed. The lozenges are then cooled, subjected to a visual check and packed into suitable packaging.

One form of suitable packaging is a blister pack of a water-impermeable plastics material (e.g., polyvinylchloride) closed by a metallic foil. The patient removes the lozenge by applying pressure to the blister to force the lozenge to rupture and pass through the metal foil seal. Lozenges will normally be sucked by the patient to release the drug. Masticable solid dose formulations may be made by the methods used to prepare chewable candy products or chewing gums. For example, a chewable solid dosage form may be prepared from an extruded mixture of sugar and glucose syrup to which the drug has been added with optional addition of whipping agents, humectants, lubricants, flavors and colorings. See Pharmaceutical Dosage Forms: Tablets, Vol. 1, Second Ed., ed. H A Lieberman, L Lachman and J B Schwartz, 1989.

U.S. Pat. No. 3,085,942 discloses the formation of an antitussive composition using dextromethorphan and its acid addition salts adsorbed, in part, on magnesium trisilicate. The inventors note that particle size of the magnesium trisilicate is not critical in preparing the adsorbates and that average particle sizes of about 0.1 to about 150 microns are usable, and also note that when the ingredients are intimately mixed, the bitter taste associated with dextromethorphan is reduced or eliminated. The adsorbate may be mixed with other ingredients to form compressed tablets, candy lozenges, chewing gum tablets and the like.

U.S. Pat. No. 4,647,459 describes an improvement where it was unexpectedly discovered that use of a particular magnesium trisilicate results in the formation of an adsorbate having adsorbency potentials greater than commercially available grades of magnesium silicates and achieves optimum medicament taste-masking characteristics while providing rapid bioavailability. There, the inventors found that an adsorbate exhibiting unexpected results is only achieved with a magnesium trisilicate having a critical surface area of at least 400 m²/g when the particles of the magnesium trisilicate exhibit a flake-like structure having multiple interstitial spaces.

U.S. Pat. No. 6,166,083 discloses the use of flurbiprofen in the treatment of sore throats which comprises the administration to a patient in need of such treatment of a pharmaceutical composition in the form of a masticable or suckable solid dosage form or a spray containing a therapeutically effective amount of flurbiprofen which releases the flurbiprofen in the oral cavity so as to deliver the flurbiprofen to the surface of the sore throat.

iv. Dissolvable Strips

Another oral delivery system suitable for use in accordance with the present invention is a dissolvable strip. An example of such a device is the Cool Mint Listerine PocketPaks® Strips, a micro-thin starch-based film impregnated with ingredients found in Listerine® Antiseptic (Thymol, Eucalyptol, Methyl Salicylate, Menthol). Non-active strip ingredients include pullulan, flavors, aspartame, potassium acesulfame, copper gluconate, polysorbate 80, carrageenan, glyceryl oleate, locust bean gum, propylene glycol and xanthan gum.

B. Media and Binders

Sterile water may be used as the diluent for glutamine. Obviously, if this is the only (or primary) diluent, it will not be possible to establish a solid or semi-solid state at or near room temperature. In such cases, the formulation will need to be subjected to low temperatures. The appropriate temperature will depend upon the freezing point of the formulation.

Another suitable media or additive is a dairy-based media such as pudding. The use of pudding has several advantages. First, it provides a base flavor. Second, the texture will help to obscure any undissolved drug. And third, it will somewhat raise the freezing point of the formulation, i.e., it acts as a binder of the media and drug. A variety of preformed puddings and instant pudding mixes are commercially available. In non-frozen formulations, gelatin may be employed.

C. Flavor Agents

Use of flavor agents and other taste modifying agents in medicinal and pharmaceutical chemistry is well known. The following is a partial list of various flavorings that are commercially available from Flavors of North America (Carol Stream, Ill.):

Internal Analgesics - Aspirin, Apap, Ibuprofen, etc.

| | | | |
|---|---|---|---|
| Mixed Berry | Fruit Punch | Wild Cherry | Orange-Cream |
| Cherry-Anise | Raspberry | Cool Cherry | Cinnamon |
| Vanilla Mint | Cool Citrus | | |

Systemic & Non-Systemic Antacids

| | | | |
|---|---|---|---|
| Bavarian Cream | Peppermint Cream | Cherry Cream | Spearmint Cream |
| Citrus Cream | Strawberry Cream | Lemon Mint | Swiss Cream |
| Lemon Cream | Vanilla Mint | Mint Cream | |

Chewable Multi-Vitamins (Plain & Fortified)

| | | | |
|---|---|---|---|
| Cherry | Orange | Citrus Punch | Raspberry |
| Cola | Strawberry | Fruit Punch | Tangerine |
| Grape | Tutti-Frutti | Lemon | |

Cough/Cold Preparations

| | | | |
|---|---|---|---|
| Anise-Menthol | Grape | Berry | Honey-Lemon |
| Cherry | Lemon | Cherry-Cinnamon | Menthol Eucalyptus |
| Cool Cherry | Orange | Fruit Punch | Raspberry |

Medicated Lozenges

| | | | |
|---|---|---|---|
| Cool Cherry | Lemon Cinnamon-Orange | Mint | Cooked |
| Fruit Punch | Menthol | Eucalyptus | Honey |

Sweetness can be used to combat bitterness. Use of aspartame, Acesulfame K, Saccharin or glycyrrhizinates can impart additional sweetness and mask a bitter taste. There are other ingredients that can be added to enhance sweetness, such as Sweet Am (A Natural Flavor), Natural Flavor Enhancer in Protein Mixes, Natural Flavor Blend, Fruit Fortifier in Beverages, Sweetness Enhancer, Artificial in Tablets, and Sweet Am (Natural and Artificial) in UHT Beverages.

Another type of flavoring is a coolness agent. Some are designed for a specific mint, such as peppermint or spearmint, while others are non-specific and even non-characterizing of mint blends. The physiology involved is not merely to numb the taste buds, but to achieve a cooling effect that builds up after ingestion. The brain perceives the coolness even though physically the temperature of the product has not changed. Traditionally, menthol has been the most recognized cooling ingredient. However, menthol can be harsh, pungent, and irritating to mucus in the mouth and nose. The following options are either non-mentholated or low in menthol concentration, and include NOR-CAP Cooling Flavor, artificial in chewing gum, Cooling Flavor (A Natural Flavor) in mouthwash, Cool Mouthfeel Flavor (A Natural and Artificial Flavor) in toothpaste, Cooling Flavor, artificial in pharmaceutical preps, and Cooling Flavor, artificial in chewing gum.

D. Anesthetics

Various topical anesthetics may be used in combination with the present invention. The following is non-limiting list of suitable anesthetics: Ametop, Anesthall, Anodyne, Athesia B, Assist II, Betacaine, Biofreeze, Dermine B, ELA-Max, ELA-Max 5, EMLA, Eutectic LA, Hurricaine, Laracaine, Laracaine, Liquidcaine, Medigel, Mentokaine B, Painless 400, Prepcaine, Prestocaine, Protocaine B, Sustaine, TAELA504, Topicaine, Trio LTB, Ultradyne, Xylocaine and Zap.

E. Unit Dosage Forms

The present invention also contemplates the use of discrete unit dosages formulations for periodic or sustained release of L-glutamine. The typical daily dose of L-glutamine for mucositis is about 20 g to about 40 g per day, though smaller doses (4-8 g/day) have been utilized. In accordance with the present invention, a lollipop, lozenge or popsicle may provide an entire daily dose, or any fractional daily dose, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 36, 37, 38, 39 or 40 g.

F. Other Ingredients

Aqueous compositions for use in preparing drug formulations of the present invention comprise L-glutamine dissolved or dispersed in a pharmaceutically acceptable medium. The phrase “pharmaceutically acceptable” refers to compositions that do not produce adverse, allergic, or other untoward reactions when administered to an animal or a human. The use of such media and agents for pharmaceutically active substances is well known in the art.

Except insofar as any conventional media or agent is incompatible with L-glutamine, its use in therapeutic compositions is contemplated. Supplementary ingredients that can be incorporated into the compositions, provided they do not inactivate L-glutamine, include antibacterial and antifungal agents, preservatives, buffering agents and the like acceptable for use in formulating pharmaceuticals.

Appropriate media may contain, for example, polyol (for example, glycerol, propylene glycol, and liquid polyethylene glycol, and the like), suitable mixtures thereof, and vegetable oils. The proper rigidity can be regulated, for example, by the use of a coating, such as lecithin, by varying the amount of aqueous diluent, by altering the gelatin, by storing at lower temperatures, or combinations thereof.

V. Examples

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

An exemplary formulation of the present invention comprises 5 g of L-glutamine mixed in Hunts® Vanilla Pudding. A 30 cc syringe is used to pull up 25 cc of pudding and squirt the pudding into the large glass beaker. A 10 cc syringe is used to pull up 5 cc of sterile water and squirt into a 500-mL beaker with the pudding. One level scoop of L-glutamine (5 g) is added to the beaker and mixed into water and pudding until the mixture is smooth without any visible clumps. A ¼ teaspoonful of Orange Kool-Aid® or ¾ teaspoonfuls of Grape Kool-Aid® is added for flavoring (per 25 cc pudding, 5 cc water, and 1 scoop of L-glutamine). The mixture is poured into a popsicle mold (this amount will fill one popsicle mold container—therefore, one may use 50 cc pudding, 10 cc water, and 2 scoops of Glutamine to fill 2 popsicles molds at a time). The entire mold is placed in a freezer (15° F.) overnight. The popsicle mold is removed from the freezer the next morning and warm water is run over the bottom of the mold for 5 minutes. The metal lid is removed from the mold. Individual popsicles are removed by gently moving the popsicle stick back and forth while pulling back on the stick at the same time. Each popsicle is placed in an individual plastic bag and put back into the freezer for storage until further use.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

VI. References

The following references, to the extent that they provide exemplary procedural or other details supplementary to those set forth herein, are specifically incorporated herein by reference:

U.S. Pat. No. 3,085,942

U.S. Pat. No. 4,647,459

U.S. Pat. No. 4,671,953

U.S. Pat. No. 5,484,602

U.S. Pat. No. 6,159,492

U.S. Pat. No. 6,166,083

Pharmaceutical Dosage Forms: Tablets, Vol. 1, Second Ed., Lieberman et al. (Eds.), 1989

What is claimed is:

1. A drug formulation designed for oral release comprising:
(a) L-glutamine; and
(b) a solid or semi-solid edible medium that dissolves upon oral contact.

2. The formulation of claim 1, provided in a unit dose of about 20 to about 40 grams L-glutamine.

3. The formulation of claim 1, further comprising a flavor agent, an antiseptic or an anesthetic.

4. The formulation of claim 1, wherein the medium is water.

5. The formulation of claim 1, wherein the medium is a gel or pudding.

6. The formulation of claim 1, wherein the medium is a mixture of water and a gel or pudding.

7. The formulation of claim 1, provided as a lozenge, a dissolvable strip, a lollipop or a popsicle.

8. The formulation of claim 1, wherein said medium dissolves in response to an aqueous environment.

9. The formulation of claim 1, wherein said medium dissolves at or near normal body temperature.

10. The formulation of claim 1, wherein said medium dissolves in response to a combination of an aqueous environment and temperature at or near normal body temperature.

11. The formulation of claim 1, provided as a popsicle comprising pudding, water, and a flavor agent.

12. The formulation of claim 1, solid at a temperature below about 0° C.

13. A method of treating oral inflammation comprising administering to a subject in need thereof a drug formulation designed for oral release comprising:
(a) L-glutamine; and
(b) a solid or semi-solid edible medium that dissolves upon oral contact.

14. The method of claim 13, wherein the formulation further comprises a flavor agent, an antiseptic or an anesthetic.

15. The method of claim 13, wherein the medium is water, gel, pudding or a mixture of water and gel or pudding.

16. The method of claim 13, wherein the formulation is provided as a lozenge, a dissolvable strip, a lollipop or a popsicle.

17. The method of claim 13, wherein said medium dissolves in response to an aqueous environment, at or near normal body temperature, or both.

18. The method of claim 13, wherein the formulation is provided as a popsicle comprising pudding, water, and a flavor agent.

19. The method of claim 13, wherein the formulation is solid at a temperature below about 0° C.

20. The method of claim 13, wherein the oral inflammation is chemotherapy-induced or radiation-induced.

21. A storage stable drug formulation comprising L-glutamine frozen in a flavored medium.

22. The formulation of claim 21, wherein the L-glutamine is stable for about 3 to about 12 months.

23. The formulation of claim 21, provided in a unit dose of about 20 to about 40 grams L-glutamine.

24. A kit comprising:
(a) L-glutamine; and
(b) a flavor agent.

25. The kit of claim 24, further comprising one or more of:
(c) an edible medium;
(d) an anesthetic;
(e) a handle; and/or
(f) a mold.
